(19) United States
(12) Patent Application Publication
(10) Pub. No.: US 2008/0045512 A1
(43) Pub. Date: Feb. 21, 2008

(54) Benzoate salt of 4-(5-methyl-oxazolo[4,5-b]-pyridin-2-yl)-1,4-diazabicyclo[3.2.2]nonane

(75) Inventors: Allen J. Duplantier, Ledyard, CT (US); Kenneth George Kraus, Old Lyme, CT (US); Bruce Nelsen Rogers, Mystic, CT (US)

Correspondence Address: PFIZER INC;Steve T. Zelson, 150 EAST 42ND STREET, 5TH FLOOR - STOP 49, NEW YORK, NY, 10017-5612, US

(73) Assignee: Pfizer Inc.

(21) Appl. No.: 11/501,289

(22) Filed: Aug. 9, 2006

(51) Int. Cl. G06F 15/00; A61K 31/5513; C07D 491/02

(52) U.S. Cl. 514/221; 540/579

(57) ABSTRACT

The present invention provides a benzoate salt of Formula I:

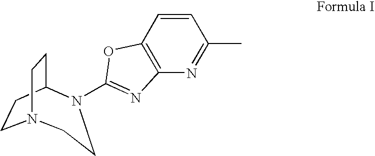

Formula I is also known as 4-(5-methyloxazolo[4,5-b]pyridine-2-yl)-1,4-diazabicyclo[3.2.2]nonane. The benzoate salt of the invention is useful in the treatment of schizophrenia and Alzheimer's Disease. It is particularly of use in the treatment of cognitive deficits associated with schizophrenia, cognitive and attention deficit symptoms of Alzheimer's Disease, and neurodegeneration associated with Alzheimer's Disease.

### FIELD OF THE INVENTION

The present invention relates to a benzoate salt of 4-(5-methyloxazolo[4,5-b]pyridine-2-yl)-1,4-diazabicyclo[3.2.2]nonane, and to a method for treating disorders of the Central Nervous System (CNS) and other disorders in a mammal, including a human, by administering to the mammal the benzoate salt. It also relates to pharmaceutical compositions containing a pharmaceutically acceptable carrier and the benzoate salt. The benzoate salt of the invention is useful in the treatment of schizophrenia and Alzheimer's Disease. It is particularly of use in the treatment of cognitive deficits associated with schizophrenia, cognitive and attention deficit symptoms of Alzheimer's Disease, and neurodegeneration associated with Alzheimer's Disease.

### BACKGROUND OF THE INVENTION

Nicotinic acetylcholine receptors (nAChRs) play a large role in central nervous system (CNS) activity and in different tissue throughout the body. They are known to be involved in functions, including, but not limited to, cognition, learning, mood, emotion, and neuroprotection. There are several types of nicotinic acetylcholine receptors, and each one appears to have a different role. Some nicotinic receptors regulate CNS function, including, but not limited to, attention, learning and memory; some regulate pain, inflammation, cancer, and diabetes by controlling tumor necrosis factor alpha (TNF-α). Nicotine affects all such receptors, and has a variety of activities. Unfortunately, not all of the activities are desirable. In fact, undesirable properties of nicotine include its addictive nature and the low ratio between efficacy and safety.

Schizophrenia is a complex multifactorial illness caused by genetic and non-genetic risk factors that produce a wide variety of symptoms. Historically, the disease has been characterized by positive and negative symptoms. The positive symptoms include delusions and hallucinations and the negative symptoms include apathy, withdrawal, lack of motivation and pleasure. More recently, deficits in affect, attention, cognition and information processing have been recognized as key pathologies in this complex disorder. No single biological element has emerged as a dominant pathogenic factor in this disease. Indeed, it is likely that schizophrenia is a syndrome that is produced by the combination of many low penetrance risk factors. Pharmacological studies established that dopamine receptor antagonists are efficacious in treating the overt psychotic features (positive symptoms) of schizophrenia such as hallucinations and delusions. Clozapine, an “atypical” antipsychotic drug, is novel because it is effective in treating not only the positive symptoms, but also negative, and to some extent the cognitive symptoms of this disease. Clozapine's utility as a drug is greatly limited because continued use leads to an increased risk of agranulocytosis and seizure. No other antipsychotic drug is effective in treating the cognitive symptoms of schizophrenia. This is significant because the restoration of cognitive functioning is the best predictor of a successful clinical and functional outcome of schizophrenic patients (Green, M. F., Am J. Psychiatry, 153:321-30, 1996).

By extension, it is clear that better drugs are needed to treat the cognitive disorders of schizophrenia in order to restore a better state of mental health to patients with this disorder. One aspect of the cognitive deficit of schizophrenia can be measured by using the auditory event-related potential (P50) test of sensory gating. In this test, electroencepholographic (EEG) recordings of neuronal activity of the hippocampus are used to measure the subject's response to a series of auditory “clicks” (Adler, L. E. et. al., Biol. Psychiatry, 46:8-18, 1999). Normal individuals respond to the first click with greater degree than to the second click. In general, schizophrenics and schizotypal patients respond to both clicks nearly the same (Cullum, C. M. et. al., Schizophr. Res., 10:131-41, 1993). These data reflect a schizophrenic's inability to “filter” or ignore unimportant information. The sensory transiently gating deficit appears to be one of the key pathological features of this disease (Cadenhead, K. S. et. al., Am. J. Psychiatry, 157:55-9, 2000). Multiple studies show that nicotine normalizes the sensory deficit of schizophrenia (Adler, L. E. et. al., Am. J. Psychiatry, 150:1856-61, 1993). Pharmacological studies indicate that nicotine's effect on sensory gating is via the α7 nAChR (Adler, L. E. et. al., Schizophr. Bull., 24:189-202, 1998). Indeed, the biochemical data indicate that schizophrenics have 50% fewer of α7 nAChR receptors in the hippocampus, thus giving a rationale to partial loss of α7 nAChR functionality (Freedman, R. et. al., Biol. Psychiatry, 38:22-33, 1995). Interestingly, genetic data indicate that a polymorphism in the promoter region of the α7 nAChR gene is strongly associated with the sensory gating deficit in schizophrenia (Freedman, R. et. al., Proc. Nat'l Acad. Sci. USA, 94(2):587-92, 1997; Myles-Worsley, M. et. al., Am. J. Med. Genet, 88(5):544-50, 1999). To date, no mutation in the coding region of the α7 nAChR has been identified. Thus, schizophrenics express the same α7 nAChR as non-schizophrenics. Selective α7 nAChR agonists may be found using a functional assay on FLIPR (see WO 00/73431). FLIPR is designed to read the fluorescent signal from each well of a 96 or 384 well plate as fast as twice a second for up to 30 minutes. This assay may be used to accurately measure the functional pharmacology of α7 nAChR. To conduct such an assay, one uses cell lines that express functional forms of the α7 nAChR using the α7/5-HT₃ channel as the drug target and cell lines that express functional 5HT₃R. In both cases, the ligand-gated ion channel was expressed in SH-EP1 cells. Both ion channels can produce robust signal in the FLIPR assay.

Bray, C, et al., “Mice Deficient in CHRNA7, a Subunit of the Nicotinic Acetylcholine Receptor, Produce Sperm with Impaired Motility”, Biol. Reprod. Jun. 8, 2005, report genetic evidence that sperm nicotinic acetylcholine receptors are important for maintenance of normal sperm motility.

Metz, Christine N., et al., 6 Nature Immunol. No 8, 756-757, 2005, and de Jonge, Wouter J., 6 Nature Immunol. No. 8., 844-851, 2005, report that acetylcholine released by stimulation of the vagus nerve binds to alpha 7 nAChRs expressed by macrophages to suppress proinflammatory cytokine production. The authors indicate that the anti-inflammatory pathway can be manipulated with cholinaergic agonists such as nicotine, providing possible therapeutic approaches for treating postoperative ileus or controlling host inflammatory responses during sepsis.

α7 nicotinic receptor agonists are also described in U.S. Pat. Nos. 6,809,094, and 6,881,734, both of which are incorporated by reference herein in their entirety.

Pharmaceutical compositions comprising an α7 nicotinic receptor agonist and an antipsychotic drug are described in US Published App. 2003/045540, which is incorporated by reference herein in its entirety.

The compositions of the present invention that contain an α7 nicotinic receptor agonist are useful for the treatment of cognitive deficits or impairments in schizophrenia and in Alzheimer's Disease.

### BRIEF DESCRIPTION OF THE DRAWINGS

FIG. 1 is the X-ray powder diffraction pattern of 4-(5-methyloxazolo[4,5-b]pyridin-2-yl)-1,4-diazabicyclo[3.2.2]nonane benzoate Form A.

FIG. 2 is the differential scanning calorimetry trace of 4-(5-methyloxazolo[4,5-b]pyridin-2-yl)-1,4-diazabicyclo[3.2.2]nonane benzoate Form A.

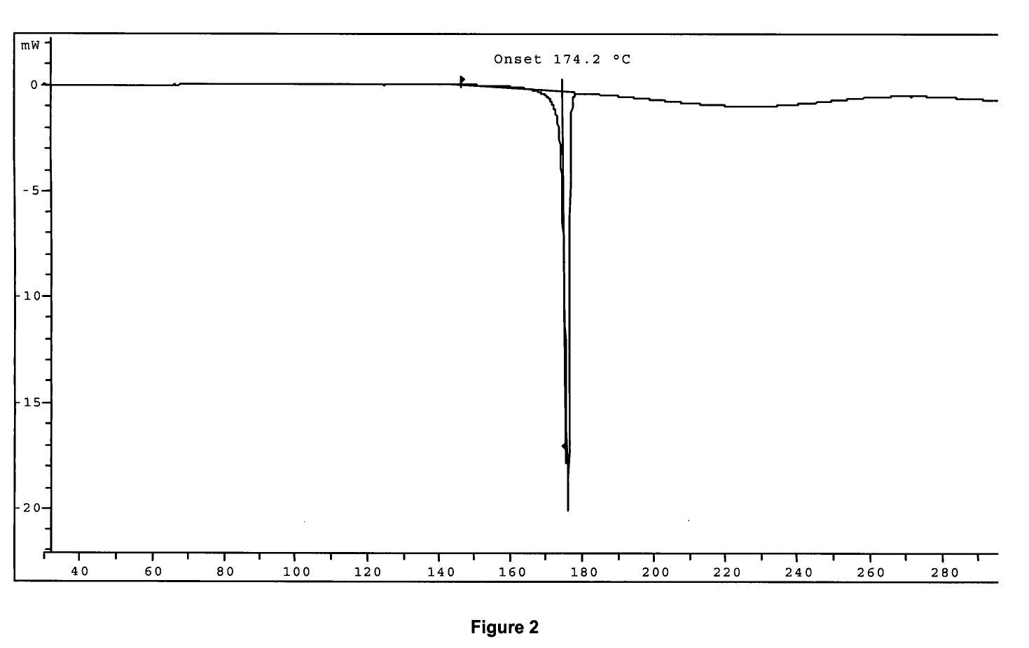

### SUMMARY OF THE INVENTION

The present invention provides a benzoate salt of Formula I:

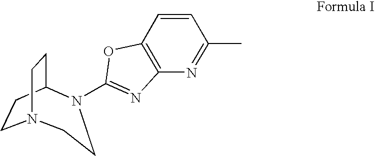

Formula I is known as 4-(5-methyloxazolo[4,5-b]pyridine-2-yl)-1,4-diazabicyclo[3.2.2]nonane, or 4-(5-methyl-oxazolo[4,5-b]pyridine-2-yl)-1,4-diaza-bicyclo[3.2.2]nonane. The benzoate salt is also known as the benzoic acid salt. The benzoate salt of Formula I of the present invention is crystalline and relatively non-hygroscopic, and exhibits physical properties that render it superior to other salts of Formula I. The benzoate salt of Formula I has been found to exist in polymorphic Form A.

The benzoate salt of the invention is useful in the treatment of schizophrenia and Alzheimer's Disease. It is particularly of use in the treatment of cognitive deficits associated with schizophrenia, cognitive and attention deficit symptoms of Alzheimer's Disease, and neurodegeneration associated with Alzheimer's Disease.

### DETAILED DESCRIPTION OF THE INVENTION

The term “treatment”, as used herein, refers to reversing, alleviating, inhibiting the progress of, or preventing the disorder or condition to which such term applies, or one or more symptoms of such condition or disorder. The term “treatment”, as used herein, refers to the act of treating, as “treating” is defined immediately above.

The benzoate salt of Formula I may exist in different polymorphic forms, all of which are encompassed by the present invention. The benzoate salt of Formula I has been found to exist in crystalline polymorphic Form A.

The benzoate salt of Formula I of the present invention encompasses solvates or hydrates thereof. The salt may form solvates or hydrates with solvents such as, but not limited to, water, acetone, and alcohol such as ethanol, propanol, butanol, propylene glycol, etc.

In an embodiment of the present invention, the benzoate salt has characteristic X-ray powder diffraction peaks as measured with copper radiation of 2-Theta±0.1° of 10.6, 12.9, 13.8, 17.6, and 19.3.

In another embodiment, the benzoate salt has characteristic X-ray powder diffraction peaks as measured with copper radiation of 2-Theta±0.1° of 10.6, 17.6, 18.6, 19.3 and 21.3.

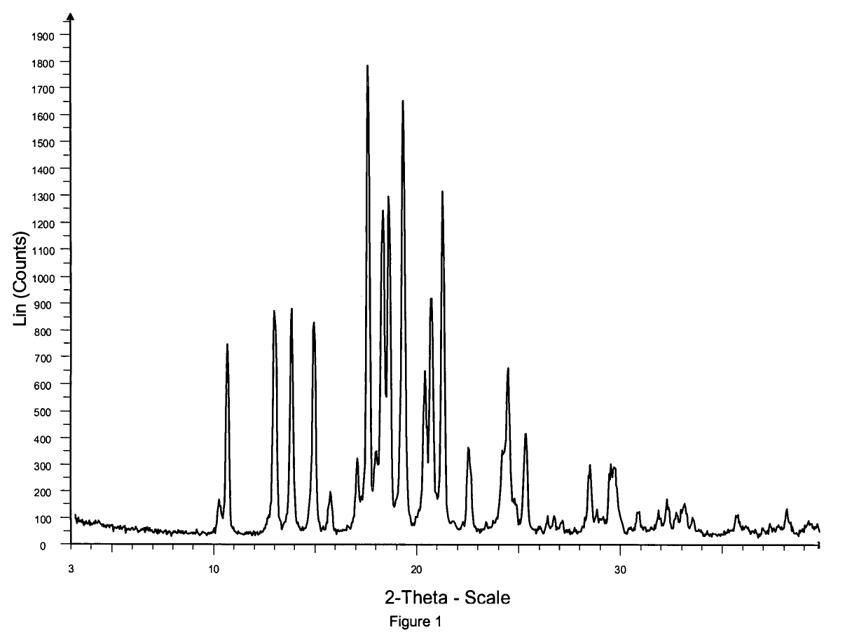

In a preferred embodiment, the benzoate salt has the characteristic X-ray powder diffraction pattern of FIG. 1.

In a further embodiment, the benzoate salt has a melting onset temperature of 174±2° C.

In another embodiment, the benzoate salt increases in weight by less than 0.2% at 90±2% relative humidity in an isothermal (24.9±0.1° C.) moisture sorption test conducted from approximately 1% to 90% (±2%) humidity.

The present invention relates to a pharmaceutical composition comprising the benzoate salt of the Formula I, and a pharmaceutically acceptable carrier. Preferably, the benzoate salt of the pharmaceutical composition is crystalline.

The present invention also relates to a pharmaceutical composition for the treatment of schizophrenia in a mammal, including a human, comprising an amount of a benzoate salt of the Formula I, that is effective in treating schizophrenia and a pharmaceutically acceptable carrier.

The present invention also relates to a method for treating schizophrenia in a mammal, including a human, comprising administering to said mammal an amount of a benzoate salt of the Formula I, that is effective in treating schizophrenia.

The present invention also relates to a pharmaceutical composition for the treatment of schizophrenia in a mammal, including a human, comprising an α7 nicotinic receptor agonizing amount of a benzoate salt of formula I and a pharmaceutically acceptable carrier.

The present invention also relates to a method for treating schizophrenia in a mammal, including a human, comprising administering to said mammal an α7 nicotinic receptor agonizing amount of a benzoate salt of the formula I.

This invention provides a method of treating a disorder or condition comprising as a symptom a deficiency in attention and/or cognition in a mammal, including a human, which method comprises administering to said mammal an amount of a benzoate salt of Formula I effective in treating said disorder or condition.

The phrase “deficiency in attention and/or cognition” as used herein in “disorder comprising as a symptom a deficiency in attention and/or cognition” refers to a subnormal functioning in one or more cognitive aspects such as memory, intellect, or learning and logic ability, in a particular individual relative to other individuals within the same general age population. “Deficiency in attention and/or cognition” also refers to a reduction in any particular individual's functioning in one or more cognitive aspects.

This invention further provides a method of treating a neurodegenerative disorder or condition in a mammal, including a human, which method comprises administering to said mammal an amount of a benzoate salt of Formula I effective in treating said disorder or condition.

As used herein, and unless otherwise indicated, a “neurodegenerative disorder or condition” refers to a disorder or condition that is caused by the dysfunction and/or death of neurons in the central nervous system. The treatment of these disorders and conditions can be facilitated by administration of an agent which prevents the dysfunction or death of neurons at risk in these disorders or conditions and/or enhances the function of damaged or healthy neurons in such a way as to compensate for the loss of function caused by the dysfunction or death of at-risk neurons.

A neurodegenerative disorder that can be treated according to the present invention includes, but is not limited to, Alzheimer's Disease.

The compounds of Formula I are useful to treat, or are useful to make a medicament to treat, a condition in a mammal that may be treated by administration of an α7 nicotinic acetylcholine receptor agonist. A benzoate salt of Formula I is useful to treat a mammal where the mammal receives symptomatic relief from activation of an α7 nicotinic acetylcholine receptor agonist.

For example, the present invention also relates to a pharmaceutical composition for treating a disorder or condition selected from cognitive and attention deficit symptoms of Alzheimer's, neurodegeneration associated with diseases such as Alzheimer's disease, pre-senile dementia (mild cognitive impairment), senile dementia, schizophrenia or psychosis including the cognitive deficits associated therewith, attention deficit disorder, attention deficit hyperactivity disorder (ADHD), mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, sleep disorders (including narcolepsy), chronic fatigue syndrome, jet lag, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependent drug cessation, Tourette's syndrome, glaucoma, neurodegeneration associated with glaucoma, symptoms associated with pain, pain and inflammation, chronic pain, acute pain, TNF-α related conditions, rheumatoid arthritis, rheumatoid spondylitis, muscle degeneration, osteoporosis, osteoarthritis, psoriasis, contact dermatitis, bone resorption diseases, atherosclerosis, Paget's disease, uveititis, gouty arthritis, inflammatory bowel disease, adult respiratory distress syndrome (ARDS), Crohn's disease, rhinitis, ulcerative colitis, anaphylaxis, asthma, Reiter's syndrome, tissue rejection of a graft, ischemia reperfusion injury, stroke, multiple sclerosis, cerebral malaria, sepsis, septic shock, toxic shock syndrome, fever and myalgias due to infection, HIV-1, HIV-2, and HIV-3, cytomegalovirus (CMV), influenza, adenovirus, a herpes virus (including HSV-1, HSV-2), a herpes zoster, cancer (multiple myeloma, acute and chronic myelogenous leukemia, or cancer-associated cachexia), diabetes (pancreatic beta cell destruction, or type I and type II diabetes), wound healing (healing burns, and wounds in general including from surgery), bone fracture healing, ischemic heart disease, tinnitus, or stable angina pectoris in a mammal, comprising an amount of a benzoate salt of the Formula I, that is effective in treating such disorder or condition and a pharmaceutically acceptable carrier. Some of the conditions that are preferred for treatment in accordance with the present invention are attention deficit disorder, attention deficit hyperactivity disorder, mood and affective disorders, cognitive deficits associated with schizophrenia, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulimia and anorexia nervosa, withdrawal symptoms associated with smoking cessation and dependent drug cessation, Gilles de la Tourette's Syndrome, glaucoma, neurodegeneration associated with glaucoma, and symptoms associated with pain.

The present invention also relates to a pharmaceutical composition for treating male infertility.

The present invention also relates to a pharmaceutical composition for treating inflammation, for example, postoperative ileus.

The present invention also relates to a method for treating a disorder or condition listed, comprising administering to a mammal in need of such treatment an amount of a benzoate salt of the Formula I, that is effective in treating such disorder or condition.

The present invention also relates to a pharmaceutical composition, which may be a composition for treating a disorder or condition listed in the previous paragraphs, comprising an α7 nicotinic receptor agonizing amount of a benzoate salt of the Formula I, and a pharmaceutically acceptable carrier.

The present invention also relates to a method for treating a disorder or condition listed in the previous paragraphs, comprising administering to a mammal in need of such treatment an α7 nicotinic receptor agonizing amount of a benzoate salt of the Formula I.

The present invention also relates to a method for treating a disease or condition in a mammal in need thereof, wherein the mammal receives symptomatic relief from activation of an α7 nicotinic acetylcholine-receptor, comprising administering to a mammal in need of such treatment a benzoate salt of the Formula I. The disease or condition may be, for example, cognitive and attention deficit symptoms of Alzheimer's, neurodegeneration associated with diseases such as Alzheimer's disease, pre-senile dementia (mild cognitive impairment), or senile dementia. The disease or condition may also be, for example, schizophrenia or psychosis and related cognitive deficits associated therewith. The disease or condition may also be any of the afore-mentioned indications, for example, attention deficit disorder, attention deficit hyperactivity disorder, mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulimia and anorexia nervosa, withdrawal symptoms associated with smoking cessation and dependent drug cessation, Gilles de la Tourette's Syndrome, glaucoma, neurodegeneration associated with glaucoma, or symptoms associated with pain.

The present invention also relates to a method for treating male infertility in a mammal in need thereof comprising administering to the mammal a benzoate salt of Formula I.

The present invention also relates to a method for treating inflammation such as postoperative ileus, in a mammal in need thereof comprising administering to the mammal a benzoate salt of Formula I.

The present invention also relates to a pharmaceutical composition comprising a benzoate salt of the Formula I, and an antipsychotic drug or pharmaceutically acceptable salt thereof.

The present invention also relates to a method of treating a mammal suffering from schizophrenia or psychosis, comprising administering a benzoate salt of Formula I, in an amount that is effective in treating schizophrenia, and an antipsychotic drug or pharmaceutically acceptable salt thereof. The benzoate salt of Formula I and the antipsychotic drug may be administered together or separately. The benzoate salt of Formula I and the antipsychotic drug may be administered simultaneously or at separate intervals. When administered simultaneously the benzoate salt of Formula I and the antipsychotic drug may be incorporated into a single pharmaceutical composition. Alternatively, two separate compositions, i.e., one containing a benzoate salt of Formula I and the other containing an antipsychotic drug, may be administered simultaneously.

The antipsychotic drug may be, for example, Chlorpromazine, Fluphenazine, Haloperidol, Loxapine, Mesoridazine, Molindone, Perphenazine, Pimozide, Thioridazine, Thiothixene, or Trifluoperazine. These drugs all have an affinity for the dopamine 2 receptor. The antipsychotic drug may also be, for example, Asenapine, Ziprasidone, Olanzapine, Clozapine, Risperidone, Sertindole, Quetiapine, Aripiprazole or Amisulpride.

Certain combinations of this invention include at least two active components: an atypical antipsychotic, a prodrug thereof, a pharmaceutically acceptable salt thereof, or a pharmaceutically acceptable salt of said prodrug, and a benzoate salt of Formula I. The combinations of this invention also include a pharmaceutically acceptable vehicle, carrier or diluent.

The combinations may result in synergistic action allowing a lower dose of the atypical antipsychotic to be administered while achieving at least the same psychotropic effect as achieved with a standard dose of the atypical antipsychotic. The dosage of the atypical antipsychotic may be reduced by about 25-90%, for example, about 40-80% and typically about 50-70%. The reduction in amount of antipsychotic required will be dependent on the amount of the benzoate salt of Formula I given.

The selection of the dosage of each therapeutic agent is that which can provide relief to the patient as measured by a reduction or amelioration of symptoms associated with the disorder or condition of the patient. As is well known, the dosage of each component depends on several factors such as the potency of the selected specific compound, the mode of administration, the age and weight of the patient, the severity of the condition to be treated, and the like. Determining a dose is within the skill of the ordinary artisan. To the extent necessary for completeness, the synthesis of the components of the compositions and dosages are as described in the listed patents above or the Physicians' Desk Reference, 57th ed., Thompson, 2003 which are expressly incorporated herein by reference. Desirably, when ziprasidone is selected as the active agent, the daily dose contains from about 5 mg to about 460 mg. More preferably, each dose of the first component contains about 20 mg to about 320 mg of the ziprasidone, and even more preferably, each dose contains from about 20 mg to about 160 mg of ziprasidone. Pediatric dosages may be less such as for example in the range of about 0.5 mg to about 40 mg daily. This dosage form permits the full daily dosage to be administered in one or two oral doses, for example.

General outlines of the dosages for the atypical antipsychotics, and some preferred dosages, are provided herein. This list is not intended to be complete but is merely a guideline for any of the desired combinations of the present invention.

Olanzapine: from about 0.25 to about 100 mg, once/day; preferably, from about 1 to about 30 mg, once/day; and most preferably about 1 to about 25 mg once/day;

Clozapine: from about 12.5 to about 900 mg daily; preferably, from about 150 to about 450 mg daily;

Risperidone: from about 0.25 to about 16 mg daily; preferably, from about 2-8 mg daily;

Sertindole: from about 0.0001 to about 1.0 mg/kg daily;

Quetiapine: from about 1.0 to about 40 mg/kg given once daily or in divided doses;

Asenapine: from about 0.005 to about 60 mg total per day, given as a single dose or in divided doses;

Paliperidone: from about 0.01 mg/kg to about 4 mg/kg body weight, more preferably from about 0.04 to about 2 mg/kg body weight;

Bifeprunox.

The presently preferred atypical antipsychotic used according to the invention is ziprasidone. Ziprasidone (5-[2-[4-(1,2-benzisothiazol-3-yl)piperazin-1-yl]ethyl]-6-chloroindolin-2-one) is a benzisothiazolyl piperazine atypical antipsychotic with in vitro activity as a 5-HT_{1A} receptor agonist and an inhibitor of serotonin and norepinephrine reuptake (U.S. Pat. No. 4,831,031). The postsynaptic 5-HT_{1A} receptor has been implicated in both depressive and anxiety disorders (NM Barnes, T Sharp, 38 Neuropharmacology 1083-152, 1999). Oral bioavailability of ziprasidone taken with food is approximately 60%, half-life is approximately 6-7 hours, and protein binding is extensive.

Ziprasidone is efficacious for the treatment of patients with schizophrenia and schizomood disorders, refractory schizophrenia, cognitive impairment in schizophrenia, affective and anxiety symptoms associated with schizoaffective disorder and bipolar disorder. The drug is considered a safe and efficacious atypical antipsychotic (Charles Caley & Chandra Cooper, 36 Ann. Pharmacother. 839-51; (2002).

The present invention is useful in treating mental disorders and conditions, the treatment of which is facilitated by the administration of ziprasidone. Thus, the present invention has application where ziprasidone use is indicated as, e.g., in U.S. Pat. Nos. 6,245,766; 6,245,765; 6,387,904; 5,312,925; 4,831,031; and European EP 0901789 published Mar. 17, 1999, all of which are incorporated herein by reference.

Other atypical antipsychotics which can be used include, but are not limited to: Olanzapine, 2-methyl-4-(4-methyl-1-piperazinyl)-10H-thieno[2,3-b][1,5]benzodiazepine. Olanizapine is a known compound and is described in U.S. Pat. No. 5,229,382 as being useful for the treatment of schizophrenia, schizophreniform disorder, acute mania, mild anxiety states, and psychosis. U.S. Pat. No. 5,229,382 is herein incorporated herein by reference in its entirety;

Clozapine, 8-chloro-11-(4-methyl-1-piperazinyl)-5H-dibenzo[b,e][1,4]diazepine. Clozapine is described in U.S. Pat. No. 3,539,573, which is herein incorporated by reference in its entirety. Clinical efficacy in the treatment of schizophrenia is described (Hanes, et al., Psychopharmacol. Bull., 24, 62 (1988));

Risperidone, 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one. Risperidone and its use in the treatment of psychotic diseases are described in U.S. Pat. No. 4,804,663, which is herein incorporated by reference in its entirety;

Sertindole, 1-[2-[4-[5-chloro-1-(4-fluorophenyl)-1H-indol-3-yl]-1-piperidinyl]ethyl]imidazolidin-2-one. Sertindole is described in U.S. Pat. No. 4,710,500. Its use in the treatment of schizophrenia is described in U.S. Pat. Nos. 5,112,838 and 5,238,945. U.S. Pat. Nos. 4,710,500; 5,112,838; and 5,238,945 are herein incorporated by reference in their entireties;

Quetiapine, 5-[2-(4-dibenzo[b,f][1,4]thiazepin-11-yl-1-piperazinyl)ethoxy]ethanol. Quetiapine and its activity in assays which demonstrate utility in the treatment of schizophrenia are described in U.S. Pat. No. 4,879,288, which is herein incorporated by reference in its entirety. Quetiapine is typically administered as its (E)-2-butenedioate (2:1) salt.

Aripiprazole, 7-{4-[4-(2,3-dichlorophenyl)-1-piperazinyl]-butoxy}-3-,4-dihydro carbostyril or 7-{4-[4-(2,3-dichlorophenyl)-1-piperazinyl]-butoxy}-3,4-dihydro-2(1H)-quinolinone. Aripiprazole is an atypical antipsychotic agent used for the treatment of schizophrenia and described in U.S. Pat. No. 4,734,416 and U.S. Pat. No. 5,006,528, which are herein incorporated by reference in their entireties.

Amisulpride, which is described in U.S. Pat. No. 4,401,822. U.S. Pat. No. 4,401,822 is incorporated herein in its entirety.

Asenapine, trans-5-chloro-2-methyl-2,3,3a,12b-tetrahydro-1H-dibenz[2,3:6,7]oxepino[4,5-c]pyrrole. Preparation and use of asenapine is described in U.S. Pat. Nos. 4,145,434 and 5,763,476, the entire contents of which are incorporated herein by reference.

Paliperidone, 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one. Preparation and use of paliparidone is described, for example, in U.S. Pat. Nos. 6,320,048; 5,158,952; and 5,254,556, the entire contents of which are incorporated herein by reference.

Bifeprunox, 2-[4-[4-(5-fluoro-1H-indol-3-yl)-3,6-dihydro-1 (2H)-pyridinyl]butyl]-1H-isoindole-1,3(2H)-dione. Preparation and use of bifeprunox is described in U.S. Pat. No. 6,225,312, which is incorporated in its entirety herein.

A preferred combination is ziprasidone with a benzoate salt of Formula I of the present invention.

Hygroscopic drug substances become moist due to their affinity for moisture in the air. Highly hygroscopic or deliquescent compounds cannot be prepared satisfactorily as powders. Nonhygroscopic drug substances are preferable for preparing solid unit dosage forms.

As used herein, “crystalline” means a material that has an ordered, long range molecular structure. The degree of crystallinity of a crystal form can be determined by many techniques including, for example, powder X-ray diffraction, moisture sorption, differential scanning calorimetry, solution calorimetry, and dissolution properties.

Crystalline organic compounds consist of a large number of atoms that are arranged in a periodic array in three-dimensional space. The structural periodicity normally manifests distinct physical properties, such as sharp, explicit spectral features by most spectroscopic probes (e.g., X-ray diffraction, infrared and solid state NMR). X-ray diffraction (XRD) is acknowledged to be one of the most sensitive methods to determine the crystallinity of solids. Crystals yield explicit diffraction maxima that arise at specific angles consistent with the lattice interplanar spacings, as predicted by Bragg's law. On the contrary, amorphous materials do not possess long-range order. They often retain additional volume between molecules, as in the liquid state. Amorphous solids normally unveil a featureless XRD pattern with broad, diffuse halos because of the absence of the long range order of repeating crystal lattice.

PXRD has reportedly been used to characterize different crystal forms of organic compounds (e.g., compounds useful in pharmaceutical compositions). See, for example, U.S. Pat. No. 5,504,216 (Holohan et al), U.S. Pat. No. 5,721,359 (Dunn et al.), U.S. Pat. No. 5,910,588 (Wangnick et al.), U.S. Pat. No. 6,066,647 (Douglas et al.), U.S. Pat. No. 6,225,474 (Matsumoto et al.), U.S. Pat. No. 6,239,141 (Allen et al.), U.S. Pat. No. 6,251,355 (Murata et al.), U.S. Pat. No. 6,288,057 (Harkness), U.S. Pat. No. 6,316,672 (Stowell et al.), and U.S. Pat. No. 6,329,364 (Groleau).

Crystalline materials are preferred in many pharmaceutical applications. Crystalline forms are generally thermodynamically more stable than amorphous forms of the same substance. This thermodynamic stability is preferably reflected in the lower solubility and improved physical stability of the crystalline form. The regular packing of the molecules in the crystalline solid preferably denies the incorporation of chemical impurities. Hence crystalline materials generally possess higher chemical purity than their amorphous counterparts. The packing in the crystalline solid generally constrains the molecules to well defined lattice positions and reduces the molecular mobility that is the prerequisite for chemical reactions. Hence, crystalline solids, with very few notable exceptions, are chemically more stable than amorphous solids of the same molecular composition.

The crystalline form of the crystalline polymorph of the benzoate salt Form A of Formula I of the present invention has the distinct powder X-ray diffraction profile provided in FIG. 1. Characteristic diffraction peaks as used herein are peaks selected from the most intense peaks of the observed diffraction pattern. Preferably, the characteristic peaks are selected from about 20 of the most intense peaks, more preferably from about 10 of the most intense peaks, and most preferably from about 4 to 5 of the most intense peaks in the diffraction pattern.

Powder X-Ray Diffraction Pattern

Powder x-ray diffraction pattern was collected for 4-(5-methyloxazolo[4,5-b]pyridin-2-yl)-1,4-diazabicyclo[3.2.2]nonane benzoate Form A using a Bruker D5000 diffractometer (Madison Wis.) equipped with a copper radiation source, fixed slits (divergence 1.0 mm, antiscatter 1.0 mm, and receiving 0.6 mm) and a Kevex solid-state detector. Data was collected in the theta-two theta goniometer configuration from a flat plate sample holder at the Copper wavelength Kα₁=1.54056 and Kα₂=1.54439 from 3.0 to 40.0 degrees two-theta using a step size of 0.040 degrees and a step time of one second. X-ray tube voltage and amperage were set at 40 kV and 40 mA respectively. Data were collected and analyzed using Bruker DIFFRAC Plus software. Samples were prepared by placing them in a quartz holder. (It is noted that a Bruker D5000 diffractometer is similar in operation to Siemans model D5000.) The results are summarized in Table 1 which provides the two-theta values and relative intensities for all of the reflections (lines) that have a relative intensity greater than or equal to 8% using a smoothing width of 0.30 and a threshold of 1.0.

TABLE 1
Powder x-ray diffraction reflections for the benzoate salt Form A of
Formula I

| Angle | Intensity* |
|---|---|
| 2-Theta ± 0.1° | % |
| 10.1 | 8.7 |
| 10.6 | 41.2 |
| 12.9 | 48.9 |
| 13.8 | 49.3 |
| 14.9 | 45.8 |
| 15.7 | 10.2 |
| 17.1 | 17.4 |
| 17.6 | 100 |
| 18.0 | 19.1 |
| 18.3 | 70.3 |
| 18.6 | 72.0 |
| 19.3 | 93.1 |
| 20.4 | 36.5 |
| 20.7 | 50.9 |
| 21.3 | 73.1 |
| 22.6 | 19.5 |
| 24.2 | 19.0 |
| 24.5 | 36.3 |
| 24.8 | 8.9 |
| 25.4 | 22.5 |
| 28.6 | 15.9 |
| 29.8 | 15.4 |
| 32.4 | 8.7 |

*The relative intensity may vary depending on particle size and shape.

Differential Scanning Calorimetry (DSC)

Thermal phase transition data was collected using a Mettler Toledo DSC 822. Crimped Aluminum sample pans with a pinhole in the lid were loaded with one to two milligrams of sample and then scanned from room temperature to 300° C. at 5° C./minute. Onset temperatures are determined by baseline tangent-peak tangent method. Onset temperatures may vary depending on particle size, sample size, sample pan configuration, and heating rate.

The thermal analysis (DSC and hot stage polarized light microscopy) of 4-(5-methyloxazolo[4,5-b]pyridin-2-yl)-1,4-diazabicyclo[3.2.2]nonane benzoate Form A indicates that it has a high melting point. Melting onset=174±2° C.

Hygroscopicity

Hygroscopicity was assessed using a dynamic vapor sorption technique in which an accurately weighed sample is subjected to progressively changing water vapor pressure while simultaneously recording the weight change. The experiment is conducted isothermally at 25° C.

For 4-(5-methyloxazolo[4,5-b]pyridin-2-yl)-1,4-diazabicyclo[3.2.2]nonane benzoate Form A, less than 0.1% increase in weight was detected at 90±2% relative humidity during an isothermal (24.9±0.1° C.) moisture sorption analysis conducted from approximately 1% to 90% (±2%) humidity. Considering standard deviation, it is considered that increase in weight is less than 0.2%. The dynamic hygroscopicity data generated suggests that it is non-hygroscopic.

### EXAMPLE 1

### 4-(5-methyloxazolo[4,5-b]pyridin-2-yl)-1,4-diazabicyclo[3.2.2]nonane benzoate

A solution of benzoic acid (95 mg, 0.77 mmol) in ethanol (5 ml) at 25° C. was added to a stirred solution of 200 mg (0.77 mmol) of 4-(5-methyloxazolo[4,5-b]pyridin-2-yl)-1,4-diazabicyclo[3.2.2]nonane (prepared from 5-methyl-2-methylsulfanyl-oxazolo[4,5-b]pyridine as reported in U.S. Pat. No. 6,809,094 which is incorporated by reference in its entirety herein) in ethanol (15 ml) at 25° C. The mixture was stirred for 1 h and then concentrated under reduced pressure to a white solid. The solid was dissolved in hot toluene (10 ml) and then allowed to slowly cool to room temperature. The resulting precipitate was filtered and dried under vacuum to give 155 mg of the title compound as white prism shaped crystals. MP 174° C.; 400 MHz ¹H NMR (CD₃OD) δ 8.0 (d, 2H), 7.5 (m, 2H), 7.4 (t, 2H), 6.9 (d, 1H), 4.6 (m, 1H), 4.1 (t, 2H), 3.4 (t, 2H), 3.3 (m, 4H), 2.5 (s, 3H), 2.3 (m, 2H), 2.1 (m, 2H); elemental analysis calculated for C₁₄H₁₈N₄O.C₇H₆O₂: C, 66.30; H, 6.36; N, 14.73. Found: C, 66.22; H, 6.31; N, 14.61.

General Preparation of Salts of Formula I

A solution of counter ion (acid) at from about 20° C. to 50° C. is dissolved in a suitable solvent and added to a solution of the free base of Formula I at from about 20° C. to about 50° C. in a suitable solvent. The mixture is stirred while cooling to room temperature followed by concentration under reduced pressure. Solids are removed by filtration.

Comparison of Salts of Formula I

Physical properties of various salts of Formula I were tested and observed. Findings included that the mesylate, fumarate, di-hydrochloride (also known as bis-hydrochloride), and L-lactate salts of Formula I demonstrated undesirable hygroscopicity. Findings also included that the sulfate, phosphate, acetate, succinate, and besylate salts did not meet rational stoichiometry upon preparation.

A benzoate salt of the Formula I of the present invention (hereinafter “the active compounds”) can be administered via either the oral, transdermal (e.g, through the use of a patch), intranasal, sublingual, rectal, parenteral or topical routes. Transdermal and oral administration are preferred. The active compounds are, most desirably, administered in dosages ranging from about 0.25 mg up to about 1500 mg per day, preferably from about 0.25 to about 300 mg per day in single or divided doses, although variations will necessarily occur depending upon the weight and condition of the subject being treated and the particular route of administration chosen. However, a dosage level that is in the range of about 0.01 mg to about 10 mg per kg of body weight per day is most desirably employed. Variations may nevertheless occur depending upon the weight and condition of the persons being treated and their individual responses to said medicament, as well as on the type of pharmaceutical formulation chosen and the time period and interval during which such administration is carried out. In some instances, dosage levels below the lower limit of the aforesaid range may be more than adequate, while in other cases still larger doses may be employed without causing any harmful side effects, provided that such larger doses are first divided into several small doses for administration throughout the day.

The active compounds can be administered alone or in combination with pharmaceutically acceptable carriers or diluents by any of the several routes previously indicated. More particularly, the active compounds can be administered in a wide variety of different dosage forms, e.g., they may be combined with various pharmaceutically acceptable inert carriers in the form of tablets, capsules, transdermal patches, lozenges, troches, hard candies, powders, sprays, creams, salves, suppositories, jellies, gels, pastes, lotions, ointments, aqueous solutions, aqueous suspensions, injectable solutions, elixirs, syrups, and the like. Such carriers include solid diluents or fillers, sterile aqueous media and various non-toxic organic solvents. In addition, oral pharmaceutical compositions can be suitably sweetened and/or flavored. In general, the active compounds are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc can be used for tabletting purposes. Solid compositions of a similar type may also be employed as fillers in gelatin capsules; preferred materials in this connection also include lactose or milk sugar, as well as high molecular weight polyethylene glycols. When aqueous suspensions and/or elixirs are desired for oral administration the active ingredient may be combined with various sweetening or flavoring agents, coloring matter and, if so desired, emulsifying and/or suspending agents, together with such diluents as water, ethanol, propylene glycol, glycerin and various combinations thereof.

For parenteral administration, a solution of an active compound in a pharmaceutically acceptable oily or aqueous vehicle such as but not limited to sesame oil, peanut oil or aqueous propylene glycol, can be employed. The aqueous solutions should be suitably buffered, if necessary, and the liquid diluent first rendered isotonic. The preparation of the solutions is under sterile conditions and is readily accomplished by standard pharmaceutical techniques well known to those skilled in the art.

Parenteral administration may be by injection, including the intravenous, intraarticular, intramuscular, and subcutaneous forms. The aqueous solutions are suitable for intravenous injection purposes. The oily solutions are suitable for intraarticular, intramuscular and subcutaneous injection purposes.

It is also possible to administer the active compounds topically and this can be done by way of creams, a patch, jellies, gels, pastes, ointments and the like, in accordance with standard pharmaceutical practice.

The effectiveness of a benzoate salt of Formula I in accordance with the present invention can be assessed according to the assays reported in U.S. Pat. Nos. 6,809,094 and 6,881,734, both of which are incorporated by reference herein in their entirety. The following information is also provided.

The benzoate salt of the invention shows potency as measured by functional activation of the α7/5-HT₃ chimeric receptor, or selectivity over other ion channels, such as 5-HT₃ or the IKr channel, or a combination thereof.

The effectiveness of the active compounds in suppressing nicotine binding to specific receptor sites can be determined by the following procedure, which is a modification of the methods of Lippiello, P. M. and Fernandes, K. G. (in “The Binding of L-[³H]Nicotine To A Single Class of High-Affinity Sites in Rat Brain Membranes”, Molecular Pharm., 29, 448-54, (1986)) and Anderson, D. J. and Arneric, S. P. (in “Nicotinic Receptor Binding of ³H-Cystisine, ³H-Nicotine and ³H-Methylcarmbamylcholine In Rat Brain”, European J. Pharm., 253, 261-67 (1994)). Male Sprague-Dawley rats (200-300 g) from Charles River were housed in groups in hanging stainless steel wire cages and were maintained on a 12 hour light/dark cycle (7 a.m.-7 p.m. light period). They received standard Purina Rat Chow and water ad libitum. The rats were killed by decapitation. Brains were removed immediately following decapitation. Membranes were prepared from brain tissue according to the methods of Lippiello and Fernandez (Molec. Pharmacol., 29, 448-454, (1986)) with some modifications. Whole brains were removed, rinsed with ice-cold buffer, and homogenized at 0° in 10 volumes of buffer (w/v) using a Brinkmann Polytron™ (Brinkmann Instruments Inc., Westbury, N.Y.), setting 6, for 30 seconds. The buffer consisted of 50 mM Tris HCl at a pH of 7.5 at room temperature. The homogenate was sedimented by centrifugation (10 minutes; 50,000×g; 0° to 4° C.). The supernatant was poured off and the membranes were gently resuspended with the Polytron and centrifuged again (10 minutes; 50,000×g; 0° C. to 4° C.). After the second centrifugation, the membranes were resuspended in assay buffer at a concentration of 1.0 g/100 mL. The composition of the standard assay buffer was 50 mM Tris HCl, 120 mM NaCl, 5 mM KCl, 2 mM MgCl₂, 2 mM CaCl₂ and had a pH of 7.4 at room temperature.

Routine assays were performed in borosilicate glass test tubes. The assay mixture typically consisted of 0.9 mg of membrane protein in a final incubation volume of 1.0 mL. Three sets of tubes were prepared wherein the tubes in each set contained 50 μL of vehicle, blank, or test compound solution, respectively. To each tube was added 200 μL of [3H]-nicotine in assay buffer followed by 750 μL of the membrane suspension. The final concentration of nicotine in each tube was 0.9 nM. The final concentration of cytisine in the blank was 1 μM. The vehicle consisted of deionized water containing 30 μL of 1 N acetic acid per 50 mL of water. The test compounds and cytisine were dissolved in vehicle. Assays were initiated by vortexing after addition of the membrane suspension to the tube. The samples were incubated at 0° to 4° C. in an iced shaking water bath. Incubations were terminated by rapid filtration under vacuum through Whatman GF/B™ glass fiber filters (Brandel Biomedical Research & Development Laboratories, Inc., Gaithersburg, Md.) using a Brandel™ multi-manifold tissue harvester (Brandel Biomedical Research & Development Laboratories, Inc., Gaithersburg, Md.). Following the initial filtration of the assay mixture, filters were washed two times with ice-cold assay buffer (5 ml each). The filters were then placed in counting vials and mixed vigorously with 20 ml of Ready Safe™ (Beckman, Fullerton, Calif.) before quantification of radioactivity. Samples were counted in a LKB Wallac Rackbeta™ liquid scintillation counter (Wallac Inc., Gaithersburg, Md.) at 40-50% efficiency. All determinations were in triplicate.

Calculations:

Specific binding (C) to the membrane is the difference between total binding in the samples containing vehicle only and membrane (A) and non-specific binding in the samples containing the membrane and cytisine (B), i.e.,

Specific binding=(C)=(A)−(B).

Specific binding in the presence of the test compound (E) is the difference between the total binding in the presence of the test compound (D) and non-specific binding (B), i.e., (E)=(D)−(B).

% Inhibition=(1−((E)/(C)) times 100.

The compounds of the invention that were tested in the above assay preferably exhibit IC₅₀ values of less than 10 μM.

[¹²⁵I]-Bungarotoxin binding to α7 nicotinic receptors in GH₄Cl cells:

Membrane preparations were made for nicotinic receptors expressed in GH₄Cl cell line. Briefly, one gram of cells by wet weight were homogenized with a polytron in 25 mis of buffer containing 20 mM Hepes, 118 mM NaCl, 4.5 mM KCl, 2.5 mM CaCl₂, 1.2 mM MgSO₄, pH 7.5. The homogenate was centrifuged at 40,000×g for 10 min at 4° C., the resulting pellet was homogenized and centrifuged again as described above. The final pellet was resuspended in 20 mis of the same buffer. Radioligand binding was carried out with [¹²⁵I] alpha-bungarotoxin from New England Nuclear, specific activity about 16 uCi/ug, used at 0.4 nM final concentration in a 96 well microtiter plate. The plates were incubated at 37° C. for 2 hours with 25 μl drugs or vehicle for total binding, 100 μl [¹²⁵I] Bungarotoxin and 125 μl tissue preparation. Nonspecific binding was determined in the presence of methyllycaconitine at 1 μM final concentration. The reaction was terminated by filtration using 0.5% Polyethylene imine treated Whatman GF/B™ glass fiberfilters (Brandel Biomedical Research & Development Laboratories, Inc., Gaithersburg, Md.) on a Skatron cell harvester (Molecular Devices Corporation, Sunnyvale, Calif.) with ice-cold buffer, filters were dried overnight, and counted on a Beta plate counter using Betaplate Scint. (Wallac Inc., Gaithersburg, Md.). Data are expressed as IC50's (concentration that inhibits 50% of the specific binding) or as an apparent Ki, IC50/1+[L]/KD. [L]=ligand concentration, KD=affinity constant for [¹²⁵I] ligand determined in separate experiment.

[¹²⁵I]-Bungarotoxin binding to alpha1 nicotinic receptors in Torpedo electroplax membranes:

Frozen Torpedo electroplax membranes (100 μl) were resuspended in 213 mis of buffer containing 20 mM Hepes, 118 mM NaCl, 4.5 mM KCl, 2.5 mM CaCl₂, 1.2 mM MgSO₄, pH 7.5 with 2 mg/ml BSA. Radioligand binding was carried out with [¹²⁵I] alpha-bungarotoxin from New England Nuclear, specific activity about 16 uCi/ug, used at 0.4 nM final concentration in a 96 well microtiter plate. The plates were incubated at 37° C. for 3 hours with 25 μl drugs or vehicle for total binding, 100 μl [¹²⁵I] Bungarotoxin and 125 μl tissue preparation. Nonspecific binding was determined in the presence of alpha-bungarotoxin at 1 μM final concentration. The reaction was terminated by filtration using 0.5% Polyethylene imine treated GF/B filters on a Brandel cell harvester with ice-cold buffer, filters were dried overnight, and counted on a Beta plate counter using Betaplate Scint. Data are expressed as IC50's (concentration that inhibits 50% of the specific binding) or as an apparent Ki, IC50/1+[L]/KD. [L]=ligand concentration, KD=affinity constant for [¹²⁵I] ligand determined in separate experiment.

5-HT₃ Receptor Binding in NG-108 Cells Using 3H-LY278584:

NG-108 cells endogenously express 5-HT₃ receptors. Cells are grown in DMEM containing 10% fetal bovine serum supplemented with L-glutamine (1:100). Cells are grown to confluence and harvested by removing the media, rinsing the flasks with phosphate buffered saline (PBS) and then allowed to sit for a 2-3 minutes with PBS containing 5 mM EDTA. Cells are dislodged and poured into a centrifuge tube. Flasks are rinsed with PBS and added to centrifuge tube. The cells are centrifuged for ten minutes at 40,000×g (20,000 rpm in Sorvall SS34 rotor (Kendro Laboratory Products, Newtown, Conn.)). The supernatant is discarded (into chlorox) and at this point the remaining pellet is weighed and can be stored frozen (−80 degrees C.) until used in the binding assay. Pellets (fresh or frozen −250 mgs per 96 well plate) are homogenized in 50 mM Tris HCl buffer containing 2 mM MgCl₂ (pH 7.4) using a Polytron homogenizer (setting 15,000 rpm) for ten seconds. The homogenate is centrifuged for ten minutes at 40,000×g. The supernatant is discarded and the pellet resuspended with the Polytron in fresh ice-cold 50 mM Tris HCl containing 2 mM MgCl₂ (pH 7.4) buffer and centrifuged again. The final pellet is resuspended in assay buffer (50 mM Tris HCl buffer (pH 7.4 at 37° C. degrees) containing 154 mM NaCl,) for a final tissue concentration of 12.5 mg per mL buffer (1.25×final concentration). Incubations were initiated by the addition of tissue homogenate to 96 well polypropylene plates containing test compounds that have been diluted in 10% DMSO/50 mM Tris buffer and radioligand (1 nM final concentration of 3H-LY278584). Nonspecific binding was determined using a saturating concentration of a known potent 5-HT₃ antagonist (10 μM ICS-205930). After an hour incubation at 37° C. in a water bath, the incubation is ended by rapid filtration under vacuum through a fire-treated Whatman GF/B glass fiber filter (presoaked in 0.5% Polyethylene imine for two hours and dried) using a 96 well Skatron Harvester (3 sec pre-wet; 20 seconds wash; 15 seconds dry). Filters are dried overnight and then placed into Wallac sample bags with 10 mLs BetaScint. Radioactivity is quantified by liquid scintillation counting using a BetaPlate counter (Wallac, Gaithersburg, Md.). The percent inhibition of specific binding is calculated for each concentration of test compound. An IC50 value (the concentration which inhibits 50% of the specific binding) is determined by linear regression of the concentration-response data (log concentration vs. logit percent values). Ki values are calculated according to Cheng & Prusoff -Ki=IC50/(1+(L/Kd)), where L is the concentration of the radioligand used in the experiment and the Kd value is the dissociation constant for the radioligand determined in separate saturation experiments.

Cell-based Assay for Measuring the EC₅₀ of α7 nAChR Agonists

Construction and Expression of the α7-5HT₃ Receptor:

The cDNA encoding the N-terminal 201 amino acids from the human α7 nAChR that contain the ligand binding domain of the ion channel was fused to the cDNA encoding the pore forming region of the mouse 5HT₃ receptor as described by Eisele J L, et al., “Chimaeric nicotinic-serotonergic receptor combines distinct ligand binding and channel specificities,” Nature (1993), Dec. 2;366(6454):479-83, and modified by Groppi, et al., WO 00/73431. The chimeric α7-5HT₃ ion channel was inserted into pGS175 and pGS179 which contain the resistance genes for G-418 and hygromycin B, respectively. Both plasmids were simultaneously transfected into SH-EP1 cells and cell lines were selected that were resistant to both G-418 and hyrgromycin B. Cell lines expressing the chimeric ion channel were identified by their ability to bind fluorescent α-bungarotoxin on their cell surface. The cells with the highest amount of fluorescent α-bungarotoxin binding were isolated using a Fluorescent Activated Cell Sorter (FACS). Cell lines that stably expressed the chimeric α7-5HT₃ were identified by measuring fluorescent α-bungarotoxin binding after growing the cells in minimal essential medium containing nonessential amino acids supplemented with 10% fetal bovine serum, L-glutamine, 100 units/ml penicillin/streptomycin, 250 ng/mg fungizone, 400 μg/ml hygromycin B, and 400 μg/ml G-418 at 37° C. with 6% CO₂ in a standard mammalian cell incubator for at least 4 weeks in continuous culture.

Assay of the Activity of the Chimeric α7-5HT₃ Receptor:

To assay the activity of the α7-5HT₃ ion channel, cells expressing the channel were plated into each well of either a 96 or 384 well dish (Corning #3614) and grown to confluence prior to assay. On the day of the assay, the cells were loaded with a 1:1 mixture of 2 mM Calcium Green 1, AM (Molecular Probes) dissolved in anhydrous DMSO and 20% pluronic F-127 (Molecular Probes). This solution was added directly to the growth media of each well to achieve a final concentration 2 μM. The cells were incubated with the dye for 60 min at 37° C. and is washed with a modified version of Earle's balanced salt solution (MMEBSS) as described in WO 00/73431. The ion conditions of the MMEBSS was adjusted to maximize the flux of calcium ion through the chimeric α7-5HT₃ ion channel as described in WO 00/73431. The activity of compounds on the chimeric α7-5HT₃ ion channel was analyzed on FLIPR. The instrument was set up with an excitation wavelength of 488 nanometers using 500 milliwatts of power. Fluorescent emission was measured above 525 nanometers with an appropriate F-stop to maintain a maximal signal to noise ratio. Agonist activity of each compound was measured by directly adding the compound to cells expressing the chimeric α7-5HT₃ ion channel and measuring the resulting increase in intracellular calcium that is caused by the agonist-induced activation of the chimeric ion channel. The assay is quantitative such that concentration-dependent increase in intracellular calcium is measured as concentration-dependent change in Calcium Green fluorescence. The effective concentration needed for a compound to cause a 50% maximal increase in intracellular calcium is termed the EC₅₀.

The benzoate salt of Formula I was tested and analyzed on FLIPR. The EC₅₀ was determined to be between 10 nM and 1000 nM.

1. A benzoate salt of Formula I:

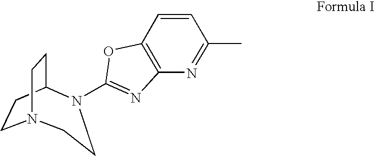

2. The salt according to claim 1, wherein the salt has characteristic X-ray powder diffraction peaks as measured with copper radiation of 2-Theta±0.1° of 10.6, 12.9, 13.8, 17.6, and 19.3.

3. The salt according to claim 1, wherein the salt has characteristic X-ray powder diffraction peaks as measured with copper radiation of 2-Theta±0.1° of 10.6, 17.6, 18.6, 19.3 and 21.3.

4. The salt according to claim 1, wherein the salt has the characteristic X-ray powder diffraction pattern of FIG. 1.

5. The salt according to claim 1, wherein the salt has a melting onset temperature of 174±2° C.

6. The salt according to claim 1, wherein the salt increases in weight by less than 0.2% at 90±2% relative humidity in an isothermal (24.9±0.1° C.) moisture sorption test conducted from approximately 1% to 90% (±2%) humidity.

7. A pharmaceutical composition comprising a benzoate salt according to claim 1, and a pharmaceutically acceptable carrier.

8. The pharmaceutical composition according to claim 7 wherein the benzoate salt is crystalline.

9. A pharmaceutical composition for the treatment of schizophrenia in a mammal, comprising an amount of a benzoate salt according to claim 1 that is effective in treating schizophrenia and a pharmaceutically acceptable carrier.

10. A method for treating schizophrenia in a mammal, comprising administering to said mammal an amount of a benzoate salt according to claim 1 that is effective in treating schizophrenia.

11. A pharmaceutical composition for the treatment of schizophrenia in a mammal, comprising an α7 nicotinic receptor agonizing amount of a benzoate salt according to claim 1 and a pharmaceutically acceptable carrier.

12. A method for treating schizophrenia in a mammal, comprising administering to said mammal an α7 nicotinic receptor agonizing amount of a benzoate salt according to claim 1.

13. A pharmaceutical composition for treating a disorder or condition selected from cognitive and attention deficit symptoms of Alzheimer's Disease, neurodegeneration associated with diseases such as Alzheimer's disease, pre-senile dementia (mild cognitive impairment), senile dementia, schizophrenia or psychosis including the cognitive deficits associated therewith, attention deficit disorder, attention deficit hyperactivity disorder (ADHD), mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependent drug cessation, Tourette's syndrome, glaucoma, neurodegeneration associated with glaucoma, symptoms associated with pain, pain and inflammation, TNF-α related conditions, rheumatoid arthritis, rheumatoid spondylitis, muscle degeneration, osteoporosis, osteoarthritis, psoriasis, contact dermatitis, bone resorption diseases, atherosclerosis, Paget's disease, uveititis, gouty arthritis, inflammatory bowel disease, adult respiratory distress syndrome (ARDS), Crohn's disease, rhinitis, ulcerative colitis, anaphylaxis, asthma, Reiter's syndrome, tissue rejection of a graft, ischemia reperfusion injury, stroke, multiple sclerosis, cerebral malaria, sepsis, septic shock, toxic shock syndrome, fever and myalgias due to infection, HIV-1, HIV-2, and HIV-3, cytomegalovirus (CMV), influenza, adenovirus, a herpes virus (including HSV-1, HSV-2), a herpes zoster, cancer (multiple myeloma, acute and chronic myelogenous leukemia, or cancer-associated cachexia), diabetes (pancreatic beta cell destruction, or type I and type II diabetes), wound healing (healing burns, and wounds in general including from surgery), bone fracture healing, ischemic heart disease, tinnitus, or stable angina pectoris comprising an amount of a benzoate salt according to claim 1, that is effective in treating such disorder or condition and a pharmaceutically acceptable carrier.

14. The composition of claim 13, wherein the disorder or condition is selected from attention deficit disorder, attention deficit hyperactivity disorder, mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependent drug cessation, Gilles de la Tourette's Syndrome, glaucoma, neurodegeneration associated with glaucoma, and symptoms associated with pain.

15. A method for treating a disorder or condition selected from attention deficit disorder, attention deficit hyperactivity disorder, mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependent drug cessation, Gilles de la Tourette's Syndrome, glaucoma, neurodegeneration associated with glaucoma, and symptoms associated with pain comprising administering to a mammal in need of such treatment an amount of a benzoate salt of Formula I, that is effective in treating such disorder or condition.

16. A method for treating a disease or condition in a mammal in need of treatment, wherein the mammal receives symptomatic relief from activation of an α7 nicotinic acetylcholine receptor, comprising administering to a mammal in need of such treatment a benzoate salt of Formula I.

17. The method of claim 16, wherein the disease or condition is selected from cognitive and attention deficit symptoms of Alzheimer's Disease, neurodegeneration associated with diseases such as Alzheimer's disease, pre-senile dementia (mild cognitive impairment), senile dementia, schizophrenia, psychosis and related cognitive deficits associated therewith, attention deficit disorder, attention deficit hyperactivity disorder, mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependent drug cessation, Gilles de la Tourette's Syndrome, glaucoma, neurodegeneration associated with glaucoma, or symptoms associated with pain.

18. The method of claim 17, wherein the disease or condition is selected from cognitive deficits associated with schizophrenia, cognitive and attention deficit symptoms of Alzheimer's Disease, and neurodegeneration associated with Alzheimer's disease.

19. A pharmaceutical composition comprising a benzoate salt of Formula I according to claim 1, and an antipsychotic drug or pharmaceutically acceptable salt thereof.

20. A method of treating a mammal suffering from cognitive impairment, schizophrenia or psychosis, comprising administering a benzoate salt of claim 1 and an antipsychotic drug or pharmaceutically acceptable salt thereof, wherein the amounts of each together are effective is treating the cognitive impairment, schizophrenia or psychosis.

21. The pharmaceutical composition of claim 19, wherein the antipsychotic drug is selected from the group consisting of Chlorpromazine, Fluphenazine, Haloperidol, Loxapine, Mesoridazine, Molindone, Perphenazine, Pimozide, Thioridazine, Thiothixene, or Trifluoperazine, Asenapine, Ziprasidone, Olanzapine, Clozapine, Risperidone, Sertindole, Quetiapine, Aripiprazole, Amisulpride, Paliperidone or Bifeprunox.

22. The method of claim 20, wherein the antipsychotic drug is selected from the group consisting of Chlorpromazine, Fluphenazine, Haloperidol, Loxapine, Mesoridazine, Molindone, Perphenazine, Pimozide, Thioridazine, Thiothixene, or Trifluoperazine, Asenapine, Ziprasidone, Olanzapine, Clozapine, Risperidone, Sertindole, Quetiapine, Aripiprazole, Amisulpride, Paliperidone or Bifeprunox.

23. The pharmaceutical composition of claim 21, wherein the antipsychotic agent is Ziprasidone.

24. The method of claim 22, wherein the antipsychotic agent is Ziprasidone.

25. A pharmaceutical composition comprising a benzoate salt of Formula I according to claim 1, and a pharmaceutically acceptable carrier, for treating male infertility.

26. A method of treating a male mammal suffering from infertility, comprising administering a benzoate salt of Formula I according to claim 1, in an amount effective in treating the infertility.

27. A pharmaceutical composition comprising a benzoate salt of Formula I according to claim 1, and a pharmaceutically acceptable carrier, for treating inflammation.

28. A method of treating a mammal suffering from inflammation, comprising administering a benzoate salt of Formula I according to claim 1, in an amount effective in treating the inflammation.
